(19) United States
(12) Patent Application Publication
(10) Pub. No.: US 2007/0185345 A1
(43) Pub. Date: Aug. 9, 2007

(54) Method for producing a benzoate

(76) Inventors: Petra Biehl, Neuss (DE); Andreas Suessenbach, Solingen (DE); Albrecht Schwerin, Duesseldorf (DE); Georg Fieg, Hamburg (DE); Heinz-Josef Krueppel, Grevenbrolch (DE); Konstantinos Scholinakis, Monheim (DE)

Correspondence Address: COGNIS CORPORATION;PATENT DEPARTMENT, 300 BROOKSIDE AVENUE, AMBLER, PA, 19002, US

(21) Appl. No.: 10/556,233

(22) PCT Filed: Apr. 30, 2004
(86) PCT No.: PCT/EP04/04589

§ 371 Date: Feb. 9, 2007

(30) Foreign Application Priority Data
May 9, 2003 (DE) 103 21 107.1

(51) Int. Cl. C07C 69/78

(52) U.S. Cl. 560/8

(57) ABSTRACT

The disclosed invention provides a process for the production of a benzoic acid ester by reaction of a benzoic acid component selected from benzoic acid or a benzoic acid ester with alcohol in the presence of a catalyst, characterized in that tin(II) oxide in combination with a phosphorus(I) compound is used as the catalyst. A phosphorus(I) acid or a salt thereof is preferably used as the phosphorus(I) compound. The resultant products can be used as constituents in cosmetic preparations.

### FIELD OF THE INVENTION

This invention relates to a process for the production of a benzoic acid ester by reaction of a benzoic acid component with alcohol in the presence of a catalyst. The invention is particularly suitable for the production of fatty acid esters of benzoic acid which are used, for example, as key substances for cosmetic preparations.

### PRIOR ART

Benzoic acid esters of the type in question are described, for example, in U.S. Pat. Nos. 4,275,222 and 4,791,097. Methanesulfonic acid or tin oxalate is used as catalyst for the reaction of the benzoic acid with the corresponding alcohol. After the esterification reaction, the product obtained has to be repeatedly washed for purification. The reactions are thus relatively complicated overall and give an unsatisfactory yield.

There was therefore a need for a process for the production of benzoic acid esters which would be simple to carry out and which would lead without additional working-up or purification steps to products of high purity which could be used inter alia in cosmetic preparations. The problem addressed by the present invention was to provide such a process.

### DESCRIPTION OF THE INVENTION

The solution to the problem stated above is provided by the process claimed in claim 1. Preferred variants of that process are described in the subsidiary claims.

In its broadest aspect, therefore, the present invention relates to a process for the production of a benzoic acid ester by reaction of a benzoic acid component selected from benzoic acid or a benzoic acid ester with alcohol in the presence of a catalyst which, according to the invention, is a combination of tin(II) oxide and a phosphorus(I) compound. Preferred phosphorus(I) compounds for the purposes of the invention are phosphorus(I) acid (i.e. phosphinic acid, hypophosphorous acid) or salts of phosphorus(I) acid (phosphinates, hypophosphites).

The catalyst used in accordance with the invention leads to high conversion levels and high yields of very pure end product. After precipitation and removal of the catalyst, the benzoic acid ester obtained can generally be used without further purification steps. The products obtained by the process according to the invention are distinguished by low acid values, water-clear colors and a faint odor. There are therefore eminently suitable for use in cosmetic preparations.

The alcohol which may be used in the esterification process according to the invention is not particularly limited. However, the process according to the invention is preferably used for the esterification of fatty alcohols or hydroxyfatty alcohols. Both natural and synthetic fatty alcohols may be used. The alcohols may be saturated or unsaturated, branched or unbranched. Preferred alcohols have a chain length of 6 to 22 carbon atoms, more particularly 8 to 18 carbon atoms and most particularly 12 to 15 carbon atoms. Mixtures of several of these alcohols may also be used. Suitable alcohol mixtures are commercially obtainable, for example, under the name of Neodol® from the Shell Chemical Company, Houston, Texas.

Preferred alcohols for the esterification process according to the invention are linear, primary alcohols. Ethoxylated and/or propoxylated fatty alcohols or even glycols, such as propylene glycol or dipropylene glycol, may be used in the process.

The alcohol is normally used in excess relative to the benzoic acid component. For example, an excess of 10 to 30% of the alcohol over the benzoic acid component is suitable.

Benzoic acid itself is used as the preferred benzoic acid component of the invention. However, a benzoic acid ester may also be used in a transesterification reaction. Esters of benzoic acid with lower alcohols, which can be distilled off from the reaction mixture, optionally under reduced pressure, during the esterification, are advantageously used. Accordingly, the methyl ester of benzoic acid is preferably used.

Particularly good results are obtained in the practical application of the process according to the invention if, in a first step (hereinafter referred to as step (A)), the reaction of the benzoic acid component with alcohol is carried out with heating under normal pressure. The reaction under normal pressure prevents the benzoic acid component from sublimating off from the reaction mixture. The educt would thus be unavailable for the esterification which would result in a reduced yield. As the reaction of the benzoic acid component progresses, there is increasingly less danger of starting material being lost through sublimation. Accordingly, at an advanced stage, the esterification may be continued at elevated temperature and reduced pressure in a second step, hereinafter referred to as step (B). Step (B) leads to completion of the esterification reaction. Once it is largely over, the esterification reaction is ended in a step (C) which is carried out in a high vacuum at elevated temperature. In this step (C), the esterification of the benzoic acid component with alcohol is continued virtually to completion and the excess alcohol is removed from the reaction mixture by distillation. The alcohol recovered can be re-used in a following reaction.

It has proved to be useful first to introduce the benzoic acid component, the alcohol and the phosphorus(I) compound and then to start the heating at the beginning of step (A) and only then to add at least part of the tin(II) oxide. The tin(II) oxide is then preferably added before the reaction mixture reaches the reaction temperature. For example, the tin(II) oxide may be added when the temperature is between two thirds of the reaction temperature and the esterification temperature. A suitable temperature range for the addition is, for example, between 150 and 190° C. and, more particularly, ca. 170° C. The reaction mixture is then heated to the esterification temperature.

The reaction in step (A) is preferably continued until the residual content of the benzoic acid component in the reaction mixture has fallen to 5% or lower. If benzoic acid itself is used as the benzoic acid component, the end point of the reaction in step (A) can also be determined on the basis of the acid value. The reaction in step (A) is preferably terminated when the acid value is below 25. This substantially corresponds to a residual acid content of 5% or less. The esterification is then continued in step (B), i.e. under reduced pressure.

Step (B) is used for esterification of the residual benzoic acid component. The pressure in the reaction vessel is preferably adjusted so that sufficient water formed or alcohol formed (methanol where benzoic acid methyl ester is the educt) distills off from the reaction mixture at the reaction temperature selected to displace the reaction equilibrium onto the product side. However, the vacuum should not be so high that relatively large quantities of benzoic acid component are removed from the reaction mixture. A vacuum of the order of ca. 200 mbar has proved to be suitable.

If only part of the tin(II) oxide was added at the start of step (A), the remaining quantity of this catalyst component is now added. The addition of the remaining tin oxide in step (B) is of advantage for achieving complete esterification. The reaction in step (B) is preferably continued until the residual content of the benzoic acid component in the reaction mixture has fallen to no more than 1%.

After step (B), the esterification reaction is completed in a high vacuum in step (C). The reduced pressure in the reaction vessel is increased to such an extent that water in the case of benzoic acid or lower alcohol in the case of a benzoic acid ester as starting product is removed substantially completely from the reaction mixture. At the same time, esterification of the benzoic acid component is also completed, so that, at the end of step (C), the acid value in the reactor is normally below 0.3 and, correspondingly, the residual content of benzoic acid component in the reaction mixture has fallen to 0.1% or lower. In other words, the esterification reaction of the process according to the invention takes place substantially completely and with virtually no loss of benzoic acid component.

After step (C) of the esterification process according to the invention, the reaction mixture is left to cool and the catalyst used in accordance with the invention is precipitated. The addition of phosphoric acid is particularly suitable for this purpose. The catalyst precipitated is then filtered off. The benzoic acid ester obtained is water-clear and has a very low acid value and a faint odor. It may be used without further purification or working up steps and, for example, may be used as such in preparations for cosmetic applications.

The quantity of catalyst used in the process according to the invention is governed by the starting components used in the esterification process. The optimal quantity for the particular esterification reaction may readily be determined by the expert. In a preferred embodiment of the process according to the invention, the quantity of tin(II) oxide used is between 0.01 and 0.6% by weight and, more particularly, 0.03 to 0.1% by weight, based on the benzoic acid component. The quantity of phosphorus(I) compound is preferably between 0.02 and 1% by weight and, more particularly, between 0.07 and 0.3% by weight, again based on the quantity of benzoic acid component used.

As already mentioned, the tin(II) oxide may be added to the reaction mixture in several stages. It has proved to be of advantage to add a large part of the tin oxide in step (A), quantities of 60 to 95% and, more particularly, between 75 and 90% of the total quantity of tin oxide having proved to be useful. The remainder is added in step (B).

The reaction temperatures are also determined to a large extent by the starting materials used. For the fatty alcohols or fatty alcohol derivatives preferably esterified in the process according to the invention, the reaction temperatures are generally between 150 and 290° C. A temperature range of 200 to 240° C. is particularly preferred. After heating of the reaction mixture in step (A), the temperature may remain substantially constant for the rest of the esterification reaction. However, the temperatures in steps (A), (B) and (C) may also be different from one another. For example, the reaction temperature may be increased during the esterification reaction in order to achieve a complete reaction of the benzoic acid component with the alcohol.

Commercial Applications

The esterification process according to the invention gives benzoic acid esters which may be used as key substances in cosmetic preparations.

The invention is illustrated by the following Example.

### EXAMPLES

### Example 1

### Production of Cetiol® AB

A mixture of primary linear alcohols with a chain length of 12 to 15 carbon atoms, which is marketed under the name of Neodol® 25E by the Shell Oil Company, Houston, Tex., is reacted in a molar excess of 25% with benzoic acid. To this end, alcohol and benzoic acid are introduced into a reactor with 0.1% by weight phosphorus(I) acid, based on the quantity of benzoic acid, at room temperature/normal pressure. The reaction mixture is gradually heated while stirring. When the reaction mixture has reached a temperature of 170° C., 0.05 part by weight tin(II) oxide, based on the quantity of benzoic acid used, is added. The reaction mixture is then heated with stirring to a reaction temperature of 220° C. The water formed during the esterification is distilled off from the reaction mixture. The reaction is continued at 220° C./normal pressure until the residual acid content in the reaction mixture has fallen below 5%. The acid value of the reaction mixture is less than 25.

After the residual acid content of less than 5% has been reached, the esterification reaction is continued at ca. 220° C. under a vacuum of ca. 200 mbar until a residual acid content in the reaction mixture of ca. 0.9% is reached.

The vacuum is then increased to less than 10 mbar in order to remove unreacted alcohol from the reaction mixture. During the removal of the residual alcohol, the acid value in the reactor falls to below 0.3 and the residual acid content reaches a value of below 0.065%.

After the unreacted alcohol has been completely removed from the reaction mixture, the reaction mixture is left to cool to room temperature and phosphoric acid is added to precipitate the catalyst from the reaction mixture. The precipitated catalyst is removed using a filter press.

The benzoic acid ester obtained is dried in vacuo. The product is water-clear, has a low acid value and only a very faint odor. It is commercially obtainable under the name of Cetiol® AB from Cognis Deutschland GmbH & Co. KG.

1-18. (canceled)

19. A process for the production of a benzoic acid ester, comprising reacting a benzoic acid component selected from benzoic acid or a benzoic acid ester with one or more alcohols in the presence of a catalyst consisting essentially of tin(II) oxide in combination with a phosphorus(I) compound.

20. The process of claim 19 wherein the phosphorus(I) compound is phosphorus(I) acid or a salt thereof.

21. The process of claim 19 wherein the one or more alcohols are selected from a fatty alcohol or a hydroxyfatty alcohol containing 6 to 22 carbon atoms.

22. The process of claim 21 wherein the fatty alcohol or a hydroxyfafty alcohol contain 12 to 15 carbon atoms.

23. The process of claim 19 wherein the one or more alcohols are linear primary alcohols.

24. The process of claim 19 wherein the one or more alcohols are ethoxylated or propoxylated fatty alcohols.

25. The process of claim 19 wherein the one or more alcohols are glycols.

26. The process of claim 19 wherein the one or more alcohols are in a molar excess of 10 to 30% over the benzoic acid component.

27. The process of claim 19 wherein the benzoic acid ester is benzoic acid methyl ester.

28. The process of claim 19 wherein the reaction of the benzoic acid component with the one or more alcohols is initially carried out with heating under normal pressure in a first step (A), subsequently continued under reduced pressure at elevated temperature in a second step (B) and then completed in a high vacuum at elevated temperature in a step (C).

29. The process of claim 28 wherein, in step (A), the benzoic acid component, the one or more alcohols and the phosphorus(I) compound are introduced first prior to heating, and at least part of the tin(II) oxide is added after the beginning of heating to the elevated temperature.

30. The process of claim 28 wherein the elevated temperature is between 150 and 190° C.

31. The process of claim 28 wherein the reaction in step (A) is continued to a residual content of the benzoic acid component in the reaction mixture of 5% or less.

32. The process of claim 28 wherein the remaining tin(II) oxide is added in step (B).

33. The process of claim 28 wherein the reaction in step (B) is continued to a residual content of the benzoic acid component of 1% or less in the reaction mixture.

34. The process of claim 28 wherein the reaction in step (C) is continued to a residual content of the benzoic acid component of 0.1% or less in the reaction mixture.

35. The process of claim 28 wherein the catalysts are precipitated after step (C) and filtered off.

36. The process of claim 35 wherein the catalyst is precipitated by addition of phosphoric acid.

37. The process of claim 1 wherein the amount of tin(II) oxide is from 0.01 and 0.6% by weight, based on the benzoic acid component, and the amount of phosphorus(I) compound is from 0.02 to 1% by weight, based on the benzoic acid component.

38. The process of claim 28 wherein 60 to 95% of the tin(II) oxide is added in step (A) and the remainder is added in step (B).

39. The process of claim 1 wherein the reaction is carried out at a temperature of 150 to 290° C.

40. A process for the production of a benzoic acid ester, comprising (A) combining a benzoic acid component selected from benzoic acid or a benzoic acid ester with one or more fatty alcohols or hydroxyfatty alcohols containing 6 to 22 carbon atoms, a phosphorus(I) co-catalyst under heating to a temperature of 150 to 190° C., after which 60 to 95% of a tin(II) oxide co-catalyst is added and the mixture heated further under normal pressure until the residual content of the benzoic acid component in the reaction mixture is 5% or less;
(B) thereafter, adding the remainder of the tin(II) oxide co-catalyst, increasing the temperature, and reducing the pressure until the residual content of the benzoic acid component in the reaction mixture is 1% or less; and
(C) further reducing the pressure to a high vacuum while maintaining the elevated temperature until an acid value below 0.3 is reached; and
(D) separating the product benzoic acid ester from the catalysts.

41. The process of claim 40 wherein the one or more alcohols are in a molar excess of 10 to 30% over the benzoic acid component claim 42 (New): The process of claim 40 wherein the reaction temperature in steps (B) and (C) is from 200 to 240° C.
